(19) United States
(12) Patent Application Publication
(10) Pub. No.: US 2024/0423899 A1
(43) Pub. Date: Dec. 26, 2024

(54) LONGEVITY MOISTURIZING CREAM

(71) Applicant: Wellbeam Consumer Health Intermediate, LLC, San Rafael, CA (US)

(72) Inventor: Jalshree Trivedi, Fremont, CA (US)

(21) Appl. No.: 18/752,564

(22) Filed: Jun. 24, 2024

Related U.S. Application Data
(60) Provisional application No. 63/509,668, filed on Jun. 22, 2023.

(51) Int. Cl. A61K 8/9789; A61K 8/34; A61Q 19/08

(52) U.S. Cl. CPC A61K 8/9789; A61K 8/342; A61K 8/345; A61Q 19/08; A61K 2800/524

(57) ABSTRACT

Cosmetic composition for skincare comprising a blend of plant-derived ingredients to reduce prevent, reduce, and treat damage to the skin. Also provided is a method of improving on or more skin condition by applying the topical compositions disclosed herein.

### CROSS REFERENCE TO RELATED APPLICATIONS

The present application claims the benefit of U.S. Provisional Patent Application No. 63/509,668, entitled, “LONGEVITY MOISTURIZING CREAM” filed Jun. 22, 2023, the contents of which is hereby incorporated by reference in its entirety.

### BACKGROUND

### 1. Field

The present inventive concept relates to topical cosmetic compositions for skincare.

### 2. Discussion of Related Art

Skin is the body's first line of defense against a range of environmental assaults including oxidative stress, ultraviolet (UV) rays, high energy visible light (violet-blue) emitted by devices such as TV/computer/smartphone screens, as well as harmful chemical agents found in airborne pollution such as smog and cigarette smoke. Additionally, skin is also subject to structural damage due to aging and other biological factors. It is therefore desirable to adequately nourish and hydrate the skin using superior ingredients.

There is a continuing need to provide compositions and methods of treatment that can improve the appearance of skin, rejuvenate skin after damage from environmental and oxidative stresses, and reduce the appearance of puffiness wrinkles and other age related symptoms on skin, especially around the eyes, while causing minimal skin irritation and side effects. These and other improvements over the art are provided by the present inventive concept.

### SUMMARY

In some aspects, the present inventive concept encompasses a topical composition, comprising, a natural retinol alternative, Coenochloris signiensis extract, at least one excipient, and at least one solvent.

In some aspects, the natural retinol alternative is present in the disclosed composition at a range of about 0.01% (wt/wt) to about 1% (wt/wt). In some aspects, the natural retinol alternative is bakuchiol.

In some aspects, the Coenochloris signiensis extract is present in the disclosed composition at a range of about 0.01% (wt/wt) to about 1% (wt/wt).

In some aspects, at least one excipient is present in the composition at about 1% (wt/wt) to about 90% (wt/wt). In some aspects of the composition, at least one excipient is cetearyl alcohol, glycerin, carbomer, xanthan gum, or any combination thereof.

In some aspects, at least one solvent is present in the disclosed composition at about 1% (wt/wt) to about 90% (wt/wt). In another aspect of the composition, at least one solvent is water.

In a further aspect, the discloses composition further comprises at least one skin conditioner and/or at least one emollient. In some aspects, at least one skin conditioner and/or at least one emollient is caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, tocopheryl acetate, or any combination thereof. In some aspects, the at least one skin conditioner and/or at least one emollient is present in the composition at about 1% (wt/wt) to about 5% (wt/wt).

In some aspects, the composition further comprises at least one preservative. In some aspects of the composition, at least one preservative is phenoxyethanol. In some aspects, at least one preservative is present in the composition at about 0.01% (wt/wt) to about 1% (wt/wt).

In some aspects, the disclosed composition further comprises at least one buffer. In some aspects, at least one buffer is sodium hydroxide. In some aspects, at least one buffer is present at in the disclosed composition at about 0.01% (wt/wt) to about 1% (wt/wt).

In some aspects, the disclosed composition further comprises at least one chelator. In some aspects, the chelator is sodium phytate. In some aspects, at least one chelator is present in the composition at about 0.01% (wt/wt) to about 1% (wt/wt).

In some aspects, the disclosed composition further comprise oils, fats, thickening agents, coloring agents, fragrance, or any combination thereof.

Further disclosed herein, is A topical composition comprising water, cetearyl alcohol, caprylic/capric triglyceride, glyceryl stearate, Cocos nucifera oil, glycerin, Aloe barbadensis leaf juice, maltodextrin, coconut alkanes, Butyrospermum parkii nut extract, bakuchiol, coco-caprylate/caprate, Coenochloris signiensis extract, lecithin, carbomer, sodium phytate, xanthan gum, ethylhexylglycerin, tocopheryl acetate, phenoxyethanol, sodium hydroxide.

The present inventive concept further encompasses topical composition comprising:
Water: about 80% (wt/wt) to about 90% (wt/wt);
Cetearyl alcohol: about 1% (wt/wt) to about 10% (wt/wt);
Caprylic/Capric Triglyceride: about 1% (wt/wt) to about 10% (wt/wt);
Glyceryl Stearate: about 1% (wt/wt) to about 10% (wt/wt);
Cocos nucifera oil: about 1% (wt/wt) to about 10% (wt/wt);
Glycerin: about 1% (wt/wt) to about 10% (wt/wt);
Aloe barbadensis leaf juice: about 1% (wt/wt) to about 10% (wt/wt);
Maltodextrin: about 1% (wt/wt) to about 10% (wt/wt);
Coconut alkanes: about 1% (wt/wt) to about 10% (wt/wt);
Butyrospermum parkii nut extract: about 1% (wt/wt) to about 10% (wt/wt);
Bakuchiol: about 0.01% (wt/wt) to about 1% (wt/wt);
Coco-Caprylate/Caprate: about 0.01% (wt/wt) to about 1% (wt/wt);
Coenochloris signiensis extract: about 0.01% (wt/wt) to about 1% (wt/wt);
Lecithin: about 0.01% (wt/wt) to about 1% (wt/wt);
Carbomer: about 0.01% (wt/wt) to about 1% (wt/wt);
Sodium phytate: about 0.01% (wt/wt) to about 1% (wt/wt);
Xanthan gum: about 0.01% (wt/wt) to about 1% (wt/wt);
Ethylhexyglycerin: about 0.01% (wt/wt) to about 1% (wt/wt);
Tocopheryl acetate: about 0.01% (wt/wt) to about 1% (wt/wt)
Phenoxyethanol: about 0.01% (wt/wt) to about 1% (wt/wt);
Sodium hydroxide: about 0.01% (wt/wt) to about 1% (wt/wt).

In some aspects, the disclosed topical composition is administered to the skin of a subject to prevent, reduce, or treat dryness, fine lines, aging of the skin cells, crow's feet, wrinkles, moisture loss, hyperpigmentation, uneven skin tone, crepe skin, collagen loss, oxidation, skin redness, roughness, free radical formation, UV damage, photo-damage, blue light damage, or any combination thereof. In some aspects, the disclosed topical composition is administered to the skin of a subject to enhance hydration, clarity, radiance, passage for absorption, skin brightness, healthy glow, even tone, elasticity, firmness, smoothness or any combination thereof.

In some aspects, the disclosed topical composition is in the form of a solids, liquids, suspensions, semisolids (such as creams, gels, pastes or “sticks”), powders or finely dispersed liquids such as sprays or mists.

In some aspects, a method of improving the condition of the skin of a subject in need thereof comprising, applying to the subject the disclosed topical composition, is further provided. In some aspects of the method, the subject is a human.

In some aspects, a kit for skincare comprising the disclosed topical composition, and instructions for use, is further provided in the present inventive concept.

### DETAILED DESCRIPTION

The following detailed description illustrates various aspects of the present inventive concept. The description is intended to describe aspects and aspects of the present inventive concept in sufficient detail to enable those skilled in the art to practice the present inventive concept. Other components can be utilized, and changes can be made without departing from the scope of the present inventive concept. The following description is, therefore, not to be taken in a limiting sense.

The present inventive concept is based at least in part on the unexpected result that a particular blend of plant-based ingredients comprising a natural retinol alternative, Coenochloris signiensis extract, and one or more excipients renew skin at the cellular level for healthy aging, slow cell aging process and protect skin against photoaging and blue light damage. The composition comprises a combination of bakuchiol, a natural retinol alternative, which reduces fine lines and even tone, Coenochloris signiensis extract, which slows aging process in skin cells, and provides hydration and smoothening to the skin. In some aspects, the composition can be formulated with other ingredients to provide additional properties, for example, suitable consistency, desired applicability profile, and other skin care effects.

### I. Compositions

In some aspects, the present inventive concept encompasses a topical composition comprising a natural retinol alternative, Coenochloris signiensis extract, at least one excipient, and at least one solvent. More specifically, the composition comprises natural retinol alternative, Coenochloris signiensis extract, at least one excipient, at least one solvent, at least one skin conditioner and/or an emollient, at least one buffer, at least one preservative, and at least one chelator. In some aspects, the ingredients used in the disclosed composition are natural, are derived from plants, are vegan, and/or are derived from non-GMO sources.

### Natural Retinol Alternative

In some aspects, the topical composition comprises a natural retinol alternative. In some aspects, the natural retinol alternative is at least one of bakuchiol, rosehip oil, rambutan, carrot seed oil, sea buckthorn oil, azelaic acid, and niacinamide. In some aspects, the natural retinol alternative in the topical composition is bakuchiol. Bakuchiol is naturally derived from the plant called Babchi and can provide similar skin care effects as retinol, without being irritating to the skin. Studies conducted on bakuchiol have determined that it is effective against multiple signs of aging. At least one study tested the effect of topical application of bakuchiol on roughness, dryness, fine lines, wrinkles, and skin tone and concluded that bakuchiol produces a significant improvement in lines and wrinkles, pigmentation, elasticity, firmness, and overall reduction in photo-damage, with no irritating effect on the skin. Further, bakuchiol was observed to strengthen the skin foundation, bring radiance, clarity, and smoothness in the skin. Studies have also shown that bakuchiol is better tolerated on the skin in topical products and provides anti-aging benefits, and overall appearance of the skin. Bakuchiol also deeply conditions and nourishes the skin. Bakuchiol can be isolated and purified from any suitable source, using known methods in the art, or can be commercially obtained (Sigma-Aldrich Catalog No. SMB00604).

In some aspects, natural retinol alternative (e.g., bakuchiol) in the disclosed topical composition reduces one or more of signs of aging on skin, roughness, dryness, fine lines, wrinkles, pigmentation, photo-damage, or any combination thereof. In some aspects, natural retinol alternative (e.g., bakuchiol) in the disclosed topical composition enhances one or more of even skin tone elasticity, firmness, smoothness, clarity, radiance, brightness, overall appearance of the skin, or any combination thereof.

In some aspects, the topical composition comprises natural retinol alternative in the amount of about 0.01% (wt/wt) to about 2% (wt/wt). In some aspects, the topical composition comprises natural retinol alternative in the amount of about 0.01% (wt/wt) to about 1% (wt/wt). In some aspects, the topical composition comprises natural retinol alternative in the amount of about 0.01% (wt/wt) to about 0.05% (wt/wt), or about 0.05% (wt/wt) to about 0.1% (wt/wt), or about 0.1% (wt/wt) to about 0.5% (wt/wt), or about 0.5% (wt/wt) to about 1% (wt/wt), or about 1% (wt/wt) to about 1.5% (wt/wt), or about 1.5% (wt/wt) to about 2% (wt/wt) by weight. In some aspects, the topical composition comprises natural retinol alternative at 0.1% (wt/wt) to about 2% (wt/wt). In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), 1% (wt/wt), 1.1% (wt/wt), 1.2% (wt/wt), 1.3% (wt/wt), 1.4% (wt/wt), 1.5% (wt/wt), 1.6% (wt/wt), 1.7% (wt/wt), 1.8% (wt/wt), 1.9% (wt/wt), or 2% (wt/wt) of natural retinol alternative. In some aspects, natural retinol alternative is present in the composition at about 0.5% (wt/wt). In some aspects, natural retinol alternative is present in the composition at about 0.01% (wt/wt). In some aspects, natural retinol alternative is present in the composition at about less than 1% (wt/wt). In some aspects, the topical composition comprises bakuchiol at an amount of about 0.5% (wt/wt). In some aspects, bakuchiol is present in the composition at about 0.01% (wt/wt). In some aspects, bakuchiol is present in the composition at about less than 1% (wt/wt).

### Coenochloris Signiensis Extract

In some aspects, In some aspects, the topical composition comprises Coenochloris signiensis extract. Clinical studies have shown that Coenochloris signiensis extract protects the skin at the cellular level and increases skin longevity resulting in increased hydration, reduced crow's feet, and slowed aging of skin cells. Coenochloris signiensis extract has been further shown to protect and activate longevity in skin cells, safeguard skin's youthfulness and, reinforce, smoothen, and hydrate the skin. Coenochloris signiensis extract can be obtained from any suitable source for e.g., commercially, from Mibelle Biochemistry Group, or Azelis Canada.

In some aspects, Coenochloris signiensis extract in the disclosed topical composition enhances skin longevity, hydration, youthfulness, smoothness, or any combination thereof. In some aspects, Coenochloris signiensis extract in the disclosed topical composition reduce crow's feet, and slows down the aging process of skin cells.

In some aspects, the topical composition comprises Coenochloris signiensis extract in the amount of about 0.01% (wt/wt) to about 5% (wt/wt). In some aspects, Coenochloris signiensis extract is present in the composition at a range of about 1% (wt/wt) to about 4% (wt/wt). In some aspects, the topical composition comprises Coenochloris signiensis extract in the amount of about 0.01% (wt/wt) to about 1% (wt/wt). In some aspects, the topical composition comprises Coenochloris signiensis extract in the amount of about 0.01% (wt/wt) to about 0.05% (wt/wt), or about 0.05% (wt/wt) to about 0.1% (wt/wt), or about 0.1% (wt/wt) to about 0.5% (wt/wt), or about 0.5% (wt/wt) to about 1% (wt/wt), or about 1% (wt/wt) to about 1.5% (wt/wt), or about 1.5% (wt/wt) to about 2% (wt/wt), or about 2% (wt/wt) to about 2.5% (wt/wt), or about 2.5% (wt/wt) to about 3% (wt/wt), or about 3% (wt/wt) to about 3.5% (wt/wt), or about 3.5% (wt/wt) to about 4% (wt/wt), or about 4% (wt/wt) to about 4.5% (wt/wt), or about 4.5% (wt/wt) to about 5% (wt/wt) by weight. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), 1% (wt/wt), 1.1% (wt/wt), 1.2% (wt/wt), 1.3% (wt/wt), 1.4% (wt/wt), 1.5% (wt/wt), 1.6% (wt/wt), 1.7% (wt/wt), 1.8% (wt/wt), 1.9% (wt/wt), 2% (wt/wt), 2.1% (wt/wt), 2.2% (wt/wt), 2.3% (wt/wt), 2.4% (wt/wt), 2.5% (wt/wt), 2.6% (wt/wt), 2.7% (wt/wt), 2.8% (wt/wt), 2.9% (wt/wt), 3% (wt/wt), 3.1% (wt/wt), 3.2% (wt/wt), 3.3% (wt/wt), 3.4% (wt/wt), 3.5% (wt/wt), 3.6% (wt/wt), 3.7% (wt/wt), 3.8% (wt/wt), 3.9% (wt/wt), 4% (wt/wt), 4.1% (wt/wt), 4.2% (wt/wt), 4.3% (wt/wt), 4.4% (wt/wt), 4.5% (wt/wt), 4.6% (wt/wt), 4.7% (wt/wt), 4.8% (wt/wt), 4.9% (wt/wt), or 5% (wt/wt) of Coenochloris signiensis extract. In a further aspect, Coenochloris signiensis extract is present in the composition at about 2% (wt/wt). In some aspects, Coenochloris signiensis extract is present in the composition at about 0.01% (wt/wt). In some aspects, Coenochloris signiensis extract is present in the composition at about less than 1% (wt/wt).

### Excipients

In some aspects, the topical compositions disclosed herein further comprise at least one excipient. In some aspects, the at least one excipient may comprise any one or more of humectants, surfactant, agents that stabilize emulsion, agents that help form gel, agents that control viscosity, oils, fats, thickening agents, coloring agents, fragrance, or any combination thereof. In some aspects, the excipient that can be used in the composition can be one or more of caprylyl glycol, ethylhexylglycerin glycerin, urea, hyaluronic acid, salicylic acid, alpha hydroxy acids (AHAs) such as glycolic acid and lactic acid, propylene glycol, honey, sorbitol, beeswax, olive oil, coconut oil, lanolin oil (isolanolin), petrolatum (petroleum jelly), mineral oil, glycerin, methyl glyceth, zinc oxide, goat milk, butyl stearate, diglycol laurate, C12-15 alkyl benzoate, aloe vera palmitate, butylene glycol, ethylhexyl palmitate, isododecane, isohexadecane, isopropyl myristate, octyldodecanol, PEG-40 hydrogenated castor oil, pentylene glycol BIO, (1, 3) propanediol, allantoin, lecithin, caprylic capric triglyceride, octyl deodecanol, cetearyl alcohol, squalene, squalene, tocopheryl acetate, polyglyceryl-6 laurate, ceteareth-20, dimethicone, PEG-100 stearate, octyldodecanol, caprylyl glycol, dicaprylyl carbonate, myristyl myristate, hectorite gel, stearyl palmitate, polyamide 3, sorbitol, polyhydroxystearic acid, lauryl laurate, cyclopentasiloxane (silicone gel), glyceryl stearate, sorbitol olivate, Jojoba esters, polysorabate 60, laureth-4, potassium cetyl sulfate, cetyl alcohol, cetearyl alcohol, stearyl alcohol, glyceryl stearate, polyglyceryl-6 laurate, ceteareth-20, sodium lauryl lactylate, myristyl myristate, carbomer, polysorbate 80, pola wax, sorbitan stearate, gum arabic, brassica alcohol, carbomer 980 QD, sodium stearate, polyhydroxystearic acid, PEG-150 distearate, glyceryl oleate, emulsifying wax, glyceryl monooleate, phospholipids, polyoxyethylene alkyl ethers, polyoxyethylene, castor oil derivatives, sorbitan fatty acid esters, polyoxyethylene stearates, polyoxylglycerides, polysorbate, sorbitan esters, sorbitan olivate, hexylene glycol, carbopol 980, cetyl alcohol, stearic acid, carnauba wax, hydroxyethyl cellulose, guar gum, xanthan gum, gelatin, magnesium aluminum silicate, cetyl silica, bentonite, palmitate, ammonium acryloyldimethyltaurate, cetearyl alcohol, glucose-D, hectorite gel, stearyl palmitate, gum arabic, hydroxypropyl starch phosphate, tapioca starch, acrylates octylacrylamide copolymer, carbomer 940, polyamide 3, castor wax, hydroxypropyl methylcellulose, caesalpinia spinosa gum, brassica alcohol, sodium stearate, polyhydroxystearic acid, tribehenin, arrowroot starch, rice starch, candelilla wax, beeswax, ozokerite wax, sunflower wax, PEG-150 distearate, polyacrylate crosspolymer-6, acrylates C10-30 alkyl acrylate cross polymer, hydroxypropyl guar, cyclopentasiloxane and (silicone gel), glycerin, propanediol, butylene glycol, ethoxydiglycol, isododecane, isohexadecane, octyldodecanol, 1,2-hexane diol, dicaprylyl carbonate, or any combination thereof.

In some aspects, at least one excipient is present in the composition at about 0.01% (wt/wt) to about 90% (wt/wt). In some aspects of the composition, the excipients comprise one or more of cetearyl alcohol, glycerin, carbomer, xanthan gum, or any combination thereof. In some aspects, the topical composition comprises cetearyl alcohol, glycerin, carbomer, and xanthan gum. In some aspects, the disclosed composition comprises one or more of cetearyl alcohol, glycerin, carbomer, xanthan gum, or any combination thereof, at about 0.01% (wt/wt) to about 90% (wt/wt). In some aspects, the disclosed composition comprises cetearyl alcohol, glycerin, carbomer, and xanthan gum, at about 0.01% (wt/wt) to about 90% (wt/wt).

In some aspects, the disclosed composition comprises cetearyl alcohol. In some aspects, the topical composition comprises about 0.01% (wt/wt) to about 15% (wt/wt) of cetearyl alcohol. In some aspects, the cetearyl alcohol is present in the disclosed composition at about 1% (wt/wt) to about 10% (wt/wt). In some aspects, the topical composition comprises about 1% (wt/wt) to about 6% (wt/wt) of cetearyl alcohol. In some aspects, the topical composition comprises about 3% (wt/wt) to about 6% (wt/wt) of cetearyl alcohol. In some aspects, the topical composition comprises cetearyl alcohol in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of cetearyl alcohol.

In some aspects, the disclosed composition comprises glycerin. In some aspects, the topical composition comprises about 0.01% (wt/wt) to about 15% (wt/wt) of glycerin. In some aspects, the glycerin is present in the disclosed composition at about 1% (wt/wt) to about 10% (wt/wt). In some aspects, the topical composition comprises about 1% (wt/wt) to about 6% (wt/wt) of glycerin. In some aspects, the topical composition comprises about 3% (wt/wt) to about 6% (wt/wt) of glycerin. In some aspects, the topical composition comprises glycerin in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of glycerin.

In some aspects, the disclosed composition comprises carbomer. In some aspects, the topical composition comprises about 0.01% (wt/wt) to about 1% (wt/wt) of carbomer. In some aspects, the topical composition comprises carbomer in the amount of about 0.01% (wt/wt) to about 0.05% (wt/wt), or about 0.05% (wt/wt) to about 0.1% (wt/wt), or about 0.1% (wt/wt) to about 0.5% (wt/wt), or about 0.5% (wt/wt) to about 1% (wt/wt). In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), 1% (wt/wt) of carbomer. In some aspects, carbomer is present in the composition at about 0.01% (wt/wt). In some aspects, carbomer is present in the composition at about less than 1% (wt/wt).

In some aspects, the disclosed composition comprises xanthan gum. In some aspects, the topical composition comprises about 0.01% (wt/wt) to about 1% (wt/wt) of xanthan gum. In some aspects, the topical composition comprises xanthan gum in the amount of about 0.01% (wt/wt) to about 0.05% (wt/wt), or about 0.05% (wt/wt) to about 0.1% (wt/wt), or about 0.1% (wt/wt) to about 0.5% (wt/wt), or about 0.5% (wt/wt) to about 1% (wt/wt). In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), 1% (wt/wt) of xanthan gum. In some aspects, xanthan gum is present in the composition at about 0.01% (wt/wt). In some aspects, xanthan gum is present in the composition at about less than 1% (wt/wt).

### Solvents

In some aspects, the disclosed composition comprises at least one solvent. In some aspects, the solvent is one or more of water, ethanol, aliphatic alcohols, esters, ethers, ketones, amines and hydrocarbons. In some aspects, the solvent is water.

In some aspects, the topical composition comprises water as a solvent. In some aspects, the topical composition comprises about 60% (wt/wt) to about 90% (wt/wt), or about 70% (wt/wt) to about 80% (wt/wt), or about 84% (wt/wt) to about 86% (wt/wt), or about 86% (wt/wt) to about 88% (wt/wt), or about 88% (wt/wt) to about 90% (wt/wt) of water. In some aspects, the composition comprises about 60% (wt/wt), about 65% (wt/wt), about 70% (wt/wt), about 75% (wt/wt), about 80% (wt/wt), about 81% (wt/wt), about 82% (wt/wt), about 83% (wt/wt), about 84% (wt/wt), about 85% (wt/wt), about 86% (wt/wt), about 87% (wt/wt), about 88% (wt/wt), about 89% (wt/wt), or about 90% (wt/wt) of water.

### Skin Conditioner and Emollient

In some aspects, the topical composition disclosed herein, further comprises at least one skin conditioner and/or at least one emollient. In some aspects, the topical composition comprises at least one skin conditioner and/or at least one emollient wherein the skin conditioner and/or emollient is one or more of caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, tocopheryl acetate, or any combination thereof. In some aspects, the topical composition comprises caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, and tocopheryl acetate.

In some aspects, at least one skin conditioner and/or at least one emollient is present in the composition at about 0.01% (wt/wt) to about 15% (wt/wt). In some aspects, one or more of caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, tocopheryl acetate, or any combination thereof, is present in the composition at about 0.01% (wt/wt) to about 15% (wt/wt). In some aspects, the topical composition comprises caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, and tocopheryl acetate at about 0.01% (wt/wt) to about 15% (wt/wt).

In some aspects, the disclosed composition comprises caprylic/capric triglyceride. In some aspects, the topical composition comprises about 0.01% (wt/wt) to about 15% (wt/wt) of caprylic/capric triglyceride. In some aspects, the caprylic/capric triglyceride is present in the disclosed composition at about 1% (wt/wt) to about 10% (wt/wt). In some aspects, the topical composition comprises about 1% (wt/wt) to about 6% (wt/wt) of caprylic/capric triglyceride. In some aspects, the topical composition comprises about 3% (wt/wt) to about 6% (wt/wt) of caprylic/capric triglyceride. In some aspects, the topical composition comprises caprylic/capric triglyceride in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of caprylic/capric triglyceride.

In some aspects, the disclosed composition comprises glyceryl stearates. In some aspects, the topical composition comprises about 0.01% (wt/wt) to about 15% (wt/wt) of glyceryl stearates. In some aspects, the glyceryl stearates is present in the disclosed composition at about 1% (wt/wt) to about 10% (wt/wt). In some aspects, the topical composition comprises about 1% (wt/wt) to about 6% (wt/wt) of glyceryl stearates. In some aspects, the topical composition comprises about 3% (wt/wt) to about 6% (wt/wt) of glyceryl stearates. In some aspects, the topical composition comprises glyceryl stearates in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of glyceryl stearates.

In some aspects, the disclosed composition comprises Cocos nucifera oil. In some aspects, the topical composition comprises about 0.01% (wt/wt) to about 15% (wt/wt) of Cocos nucifera oil. In some aspects, the Cocos nucifera oil is present in the disclosed composition at about 1% (wt/wt) to about 10% (wt/wt). In some aspects, the topical composition comprises about 1% (wt/wt) to about 6% (wt/wt) of Cocos nucifera oil. In some aspects, the topical composition comprises about 3% (wt/wt) to about 6% (wt/wt) of Cocos nucifera oil. In some aspects, the topical composition comprises Cocos nucifera oil in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of Cocos nucifera oil.

In some aspects, the disclosed composition comprises Aloe barbadensis leaf juice. In some aspects, the topical composition comprises about 0.01% (wt/wt) to about 15% (wt/wt) of Aloe barbadensis leaf juice. In some aspects, the Aloe barbadensis leaf juice is present in the disclosed composition at about 1% (wt/wt) to about 10% (wt/wt). In some aspects, the topical composition comprises about 1% (wt/wt) to about 6% (wt/wt) of Aloe barbadensis leaf juice. In some aspects, the topical composition comprises about 3% (wt/wt) to about 6% (wt/wt) of Aloe barbadensis leaf juice. In some aspects, the topical composition comprises Aloe barbadensis leaf juice in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of Aloe barbadensis leaf juice.

In some aspects, the disclosed composition comprises maltodextrin. In some aspects, the topical composition comprises about 0.01% (wt/wt) to about 15% (wt/wt) of maltodextrin. In some aspects, the maltodextrin is present in the disclosed composition at about 1% (wt/wt) to about 10% (wt/wt). In some aspects, the topical composition comprises about 1% (wt/wt) to about 6% (wt/wt) of maltodextrin. In some aspects, the topical composition comprises about 3% (wt/wt) to about 6% (wt/wt) of maltodextrin. In some aspects, the topical composition comprises maltodextrin in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of maltodextrin.

In some aspects, the disclosed composition comprises coconut alkanes. In some aspects, the topical composition comprises about 0.01% (wt/wt) to about 15% (wt/wt) of coconut alkanes. In some aspects, the coconut alkanes is present in the disclosed composition at about 1% (wt/wt) to about 10% (wt/wt). In some aspects, the topical composition comprises about 1% (wt/wt) to about 6% (wt/wt) of coconut alkanes. In some aspects, the topical composition comprises about 3% (wt/wt) to about 6% (wt/wt) of coconut alkanes. In some aspects, the topical composition comprises coconut alkanes in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of coconut alkanes.

In some aspects, the disclosed composition comprises Butyrospermum parki nut extract. In some aspects, the topical composition comprises about 0.01% (wt/wt) to about 15% (wt/wt) of Butyrospermum parki nut extract. In some aspects, the Butyrospermum parki nut extract is present in the disclosed composition at about 1% (wt/wt) to about 10% (wt/wt). In some aspects, the topical composition comprises about 1% (wt/wt) to about 6% (wt/wt) of Butyrospermum parki nut extract. In some aspects, the topical composition comprises about 3% (wt/wt) to about 6% (wt/wt) of Butyrospermum parki nut extract. In some aspects, the topical composition comprises Butyrospermum parki nut extract in the amount of about 0.1% (wt/wt) to about 15% (wt/wt), or about 0.5% (wt/wt) to about 12% (wt/wt), or about 1% (wt/wt) to about 7% (wt/wt), or about 1% (wt/wt) to about 5% (wt/wt). In some aspects, the topical composition comprises about 1.5% (wt/wt), about 1.6% (wt/wt), about 1.7% (wt/wt), about 1.8% (wt/wt), about 1.9% (wt/wt), about 2.0% (wt/wt), about 2.1% (wt/wt), about 2.2% (wt/wt), about 2.3% (wt/wt), about 2.4% (wt/wt), about 2.5% (wt/wt), about 2.6% (wt/wt), about 2.7% (wt/wt), about 2.8% (wt/wt), about 2.9% (wt/wt), about 3.0% (wt/wt), about 3.1% (wt/wt), about 3.2% (wt/wt), about 3.3% (wt/wt), about 3.4% (wt/wt), about 3.5% (wt/wt), about 3.6% (wt/wt), about 3.7% (wt/wt), about 3.8% (wt/wt), about 3.9% (wt/wt), about 4.0% (wt/wt), about 4.2% (wt/wt), about 4.3% (wt/wt), about 4.4% (wt/wt), about 4.5% (wt/wt), about 4.6% (wt/wt), about 4.7% (wt/wt), about 4.8% (wt/wt), about 4.9% (wt/wt), about 5.0% (wt/wt), about 5.1% (wt/wt), about 5.2% (wt/wt), about 5.3% (wt/wt), about 5.4% (wt/wt), about 5.5% (wt/wt), about 5.6% (wt/wt), about 5.7% (wt/wt), about 5.8% (wt/wt), about 5.9% (wt/wt), about 6.0% (wt/wt), 7% (wt/wt), 8% (wt/wt), 9% (wt/wt), 10% (wt/wt), 11% (wt/wt), 12% (wt/wt), 13% (wt/wt), 14% (wt/wt), or 15% (wt/wt) of Butyrospermum parki nut extract.

In some aspects, the disclosed composition comprises coco-caprylate/caprate. In some aspects, the topical composition comprises about 0.01% (wt/wt) to about 1% (wt/wt) of coco-caprylate/caprate. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of coco-caprylate/caprate. In some aspects, coco-caprylate/caprate is present in the composition at about 0.01% (wt/wt). In some aspects, coco-caprylate/caprate is present in the composition at about less than 1% (wt/wt).

In some aspects, the disclosed composition comprises lecithin. In some aspects, the topical composition comprises about 0.01% (wt/wt) to about 1% (wt/wt) of lecithin. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of lecithin. In some aspects, lecithin is present in the composition at about 0.01% (wt/wt). In some aspects, lecithin is present in the composition at about less than 1% (wt/wt).

In some aspects, the disclosed composition comprises ethylhexylglycerin. In some aspects, the topical composition comprises about 0.01% (wt/wt) to about 1% (wt/wt) of ethylhexylglycerin. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of ethylhexylglycerin. In some aspects, ethylhexylglycerin is present in the composition at about 0.01% (wt/wt). In some aspects, ethylhexylglycerin is present in the composition at about less than 1% (wt/wt).

In some aspects, the disclosed composition comprises tocopheryl acetate. In some aspects, the topical composition comprises about 0.01% (wt/wt) to about 1% (wt/wt) of tocopheryl acetate. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of tocopheryl acetate. In some aspects, tocopheryl acetate is present in the composition at about 0.01% (wt/wt). In some aspects, tocopheryl acetate is present in the composition at about less than 1% (wt/wt).

### Preservative

In some aspects, the topical composition further comprises at least one preservative. In some aspects, the preservative is one or more of phenoxyethanol, sodium benzoate, or any combination thereof. In some aspects, the topical composition comprises phenoxyethanol. In some aspects, the preservative is present in the composition at about 0.01% (wt/wt) to about 1% (wt/wt). In some aspects, the topical composition comprises phenoxyethanol, at about 0.01% (wt/wt) to about 1% (wt/wt).

In some aspects, the topical composition comprises phenoxyethanol. In some aspects, the topical composition comprises about 0.01% (wt/wt) to about 1% (wt/wt) of phenoxyethanol. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of phenoxyethanol. In some aspects, phenoxyethanol is present in the composition at about 0.01% (wt/wt). In some aspects, phenoxyethanol is present in the composition at about less than 1% (wt/wt).

### Buffer

In some aspects, the topical composition further comprises at least one buffer. In some aspects, the buffer is one or more of sodium hydroxide, citric acid, or any combination thereof. In some aspects, the disclosed composition comprises sodium hydroxide. In some aspects, the buffer is present in the composition at about 0.01% (wt/wt) to about 5% (wt/wt). In some aspects, the disclosed composition comprises sodium hydroxide, at about 0.01% (wt/wt) to about 5% (wt/wt).

In some aspects, the In some aspects, the topical composition comprises sodium hydroxide. In some aspects, the topical composition comprises about 0.01% (wt/wt) to about 1% (wt/wt) of sodium hydroxide. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of sodium hydroxide. In some aspects, sodium hydroxide is present in the composition at about 0.01% (wt/wt). In some aspects, sodium hydroxide is present in the composition at about less than 1% (wt/wt).

### Chelator

In some aspects, the topical composition further comprises at least one chelator. In some aspects, the chelator is one or more of sodium phytate, gluconolactone, calcium gluconate, or any combination thereof. In some aspects, the disclosed composition comprises sodium phytate. In some aspects, the chelator is present in the composition at about 0.01% (wt/wt) to about 5% (wt/wt). In some aspects, the disclosed composition comprises sodium phytate at about 0.01% (wt/wt) to about 5% (wt/wt).

In some aspects, the topical composition comprises sodium phytate. In some aspects, the topical composition comprises about 0.01% (wt/wt) to about 1% (wt/wt) of sodium phytate. In some aspects, the topical composition comprises about 0.01% (wt/wt), 0.02% (wt/wt), 0.03% (wt/wt), 0.04% (wt/wt), 0.05% (wt/wt), 0.06% (wt/wt), 0.07% (wt/wt), 0.08% (wt/wt), 0.09% (wt/wt), 0.1% (wt/wt), 0.2% (wt/wt), 0.3% (wt/wt), 0.4% (wt/wt), 0.5% (wt/wt), 0.6% (wt/wt), 0.7% (wt/wt), 0.8% (wt/wt), 0.9% (wt/wt), or 1% (wt/wt) of sodium phytate. In some aspects, sodium phytate is present in the composition at about 0.01% (wt/wt). In some aspects, sodium phytate is present in the composition at about less than 1% (wt/wt).

In some aspects, the composition further comprise oils, fats, thickening agents, coloring agents, fragrance, or any combination thereof. Such ingredients may be present in the compositions, at about 0.01% (wt/wt) to about 1% (wt/wt). In further aspects, the present composition may include one or more of other active and inactive ingredients, including, but not limited to vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, antiacne agents, anesthetics, antiseptics, insect repellents, skin cooling compounds, skin protectants, skin penetration enhancers, exfoliants, lubricants, fragrances, colorants, staining agents, depigmenting agents, hypopigmenting agents, preservatives, stabilizers, pharmaceutical agents, photostabilizing agents, and mixtures thereof. In addition to the foregoing, the composition of the disclosure may contain any other compound for the treatment of skin disorders.

In some aspects, the disclosed topical composition comprises water, cetearyl alcohol, caprylic/capric triglyceride, glyceryl stearate, Cocos nucifera oil, glycerin, Aloe barbadensis leaf juice, maltodextrin, coconut alkanes, Butyrospermum parkii nut extract, bakuchiol, coco-caprylate/caprate, Coenochloris signiensis extract, lecithin, carbomer, sodium phytate, xanthan gum, ethylhexylglycerin, tocopheryl acetate, phenoxyethanol, sodium hydroxide. In some aspects, the topical composition comprises one or more ingredients at range of amounts listed in Table 1.

TABLE 1
Topical composition

| Ingredients | Amount % (wt/wt) |
|---|---|
| Water | about 80% to about 90% |
| Cetearyl alcohol | about 1% to about 10% |
| Caprylic/capric triglyceride | about 1% to about 10% |
| Glyceryl stearate | about 1% to about 10% |
| Cocos nucifera oil | about 1% to about 10% |
| Glycerin | about 1% to about 10% |
| Aloe barbadensis leaf juice | about 1% to about 10% |
| Maltodextrin | about 1% to about 10% |
| Coconut alkanes | about 1% to about 10% |
| Butyrospermum parkii nut extract | about 1% to about 10% |
| Bakuchiol | about 0.01% to about 1% |
| Coco-caprylate/caprate | about 0.01% to about 1% |
| Coenochloris signiensis extract | about 0.01% to about 1% |
| Lecithin | about 0.01% to about 1% |
| Carbomer | about 0.01% to about 1% |
| Sodium phytate | about 0.01% to about 1% |
| Xanthan gum | about 0.01% to about 1% |
| Ethylhexyglycerin | about 0.01% to about 1% |
| Tocopheryl acetate | about 0.01% to about 1% |
| Phenoxyethanol | about 0.01% to about 1% |
| Sodium hydroxide | about 0.01% to about 1% |

In some aspects, the compositions disclosed herein may be in any suitable form including but not limited to solids, liquids, suspensions, semisolids (such as creams, gels, pastes or “sticks”), powders, or finely dispersed liquids such as sprays or mists. In some aspects, the compositions may packaged into bottles, tubes, tablets, applicators, capsules, soap, facial peel, or facial mask. In certain aspects, the composition is formulated as a cosmetic composition non-limiting examples of which include serum, solution, suspension, lotion, oil, cream, gel, toner, stick, pencil, ointment, paste, foam, powder, balm, mousse, shaving cream, wipe, strip, patch, wound dressing and adhesive bandage, hydrogel, film-forming product, facial and skin mask, cosmetics (e.g., foundations, eye liners, eye shadows, concealer, primer), and the like. In certain aspects, the composition of the present disclosure is formulated as a moisturizing cream. The composition formulated as a moisturizing cream can be a composition disclosed in any one of the Tables 1-4.

Also contemplated are containers comprising a composition of the present disclosure. In non-limiting aspects, the container can be a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The containers can optionally have spray, pump, or squeeze mechanisms.

The present composition may be prepared into a desired formulation by any known methods in the art. For example, the composition may be prepared by mixing one or more disclosed ingredients into a homogeneous product. The method comprises mixing the natural retinol alternative, and Coenochloris signiensis extract, optionally one or more additional ingredients as described herein, and water to form the skin care formulation. The mixing may be carried out, for example, by using a mixer or blender.

### II. Methods of Using

In some aspects, the present inventive concept also encompasses methods of using the compositions disclosed herein for improving one or more condition of the skin in a subject.

The method of improving one or more condition of the skin of a subject comprises applying a composition of the present disclosure to the skin of the subject, for e.g., skin around the eyes. In some aspects, the composition is applied for about 24 hours, about 20 hours, about 16 hours, about 12 hours, about 10 hours, about 8 hours, about 6 hours, about 4 hours, about 2 hours, about 1 hour or less. In further aspects, the composition is applied at least three times, at least four times, at least five times, at least six times, at least seven times, at least eight times, at least nine times, or at least ten times over a period of time (e.g., one week, two weeks, three weeks, a month, two months, three months, etc.). The method may include applying the composition a plurality of times (e.g., one, two, three, four, five, or six times) each day for a period of time (e.g., one week, two weeks, three weeks, a month, two months, three months, etc.).

In certain aspects, the method may include applying an amount of the composition such that about 0.01 gram or more is applied to the skin around the eyes. For example, the composition may be applied such that about 0.01 gram per decimeter (g/dm²) or more, about 0.5 g/dm² or more, about 07.5 g/dm² or more, about 1 g/dm² or more, about 1.5 g/dm² or more, about 2 g/dm² or more, about 2.5 g/dm² or more, or about 3 g/dm² or of the composition is applied to the skin. In some cases, the composition is applied such that about 0.1 to about 10 gram per decimeter (g/dm²), about 0.1 to about 9 g/dm², about 0.1 to about 8 g/dm², about 0.1 to about 7 g/dm², about 0.1 to about 6 g/dm², about 0.1 to about 5 g/dm², about 0.1 to about 4 g/dm², about 0.1 to about 3 g/dm², about 0.1 to about 2 g/dm², about 0.1 to about 1 g/dm²; about 0.5 to about 10 gram per decimeter (g/dm²), about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 gram per decimeter (g/dm²), about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of the composition is applied to the skin, for example skin around of the eyes.

In some aspects, the compositions disclosed herein is administered to the skin of a subject to prevent, reduce, or treat dryness, fine lines, aging of the skin cells, crow's feet, wrinkles, moisture loss, hyperpigmentation, uneven skin tone, crepe skin, collagen loss, oxidation, skin redness, roughness, free radical formation, UV damage, photo-damage, blue light damage, or any combination thereof. In one aspect, the administration of the composition disclosed herein reduce one or more of dryness, fine lines, aging of the skin cells, crow's feet, wrinkles, moisture loss, hyperpigmentation, uneven skin tone, crepe skin, collagen loss, oxidation, skin redness, roughness, free radical formation, UV damage, photo-damage, and blue light damage in the subject, by at least 10%, e.g., 20%, 30%, 40%, 50%, 60%, 70%, 80%, 90%, 95% or above, as compared to the subject before administration of the composition or a control subject not receiving the composition. The change in the skin condition can be assessed by one or more a skin care professionals, or can be undertaken by self-assessment, using, for example, image-based analysis. Images, for example facial pictures, can be taken before and after treatments, or of the control subjects, and visually compared to read the differences in skin condition. In an alternate aspect, photographs may be analyzed by a computer, using a computer-based grading using a facial computational algorithm that have been previously validated for assessment of the skin condition.

In some aspects, the disclosed topical composition is administered to the skin of a subject to enhance hydration, clarity, radiance, passage for absorption, skin brightness, healthy glow, even tone, elasticity, firmness, smoothness or any combination thereof. In one aspect, the administration of the composition disclosed herein enhance one or more of hydration, clarity, radiance, passage for absorption, skin brightness, healthy glow, even tone, elasticity, firmness, and smoothness in the skin of the subject, by at least 10%, e.g., 20%, 30%, 40%, 50%, 60%, 70%, 80%, 90%, 95% or above, as compared to the subject before administration of the composition or a control subject not receiving the composition. The change in the skin condition can be assessed by one or more skin care professionals, or can be undertaken by self-assessment, using, for example, image-based analysis. Images, for example facial pictures, can be taken before and after treatments, or of the control subjects, and visually compared to read the differences in skin condition. In an alternate aspect, photographs may be analyzed by a computer, using a computer-based grading using a facial computational algorithm that have been previously validated for assessment of the skin condition.

In some aspects, the composition may be applied to the skin one or more times daily, one or more times weekly, one or more time monthly or at any interval for any duration of time. For example, as a liquid composition, 3-5 drops of the composition may be used daily or multiple times in a day. By way of another example, as a cream or lotion composition, a thin, medium, or thick layer of the composition may be used daily or multiple times in a day. In some instances the composition may be a cream and a thin layer is used. In some aspects, such use may provide reduction in visible fine lines and wrinkles by about 1% to 20% as estimated by methods known in the art. In some aspects, use of 1, or 2, or 3, or 4, or 5, or 6, or 7, or 8, or 9, or 10 drops daily can reduce one or more of fine lines, wrinkles, moisture loss, hyperpigmentation, uneven skin tone, crepe skin, collagen loss, oxidation, uneven skin tone, skin redness, and free radical formation. In some aspects, the composition can be applied to any external or exposed part of the body. For example, the composition can be applied to the face, hands, elbow, knees, legs, toes, neck, or fingers.

In some aspects, the subject is a human. In some aspects, the subject is a human of any age from 0-120 yrs.

### III. Kits

In some aspects, the present inventive concept also encompasses kits comprising the topical composition disclosed herein and instructions for use.

### I. Terminology

The phraseology and terminology employed herein are for the purpose of description and should not be regarded as limiting. For example, the use of a singular term, such as, “a” is not intended as limiting of the number of items. The kit may include a container comprising the composition disclosed herein. The container can be a bottle, a dispenser, a package, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The container can dispense a pre-determined amount of the composition. In certain aspects, the compositions is dispensed in a spray, dollop, or liquid. The container can include indicia on its surface. The indicia can be a word, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### I. Terminology

Further, as the present inventive concept is susceptible to aspects of many different forms, it is intended that the present inventive concept be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present inventive concept, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, “and/or” refers to and encompasses any and all possible combinations of one or more of the associated listed items, as well as the lack of combinations were interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure of the present inventive concept.

The term “topical composition” or “topical formulation” or “topical cosmetics” or “topical products” as used herein encompasses substances that may be applied topically to the skin or mucous membranes of humans or animals in order to alter the subject's appearance, to protect the subject from the environment, or to produce a biological change in the skin or other tissue for therapeutic, preventive or cosmetic purposes. Topical products occur in a variety of forms, including solids, liquids, suspensions, semisolids (such as creams, gels, pastes, or “sticks”), powders, or finely dispersed liquids such as sprays or mists. Examples of topical products commonly classified as “cosmetics” include skin care products such as creams, lotions, moisturizers, serums, and “treatment cosmetics” such as exfoliants and/or skin cell renewal agents.

As used herein, “individual”, “subject”, “host”, and “patient” can be used interchangeably herein and refer to any mammalian subject, for example, humans.

The term “emollient” as used herein encompasses ingredients in skin creams, lotions, moisturizers or ointments that form a film on the skin. These ingredients can relieve dryness, itching, and scaling. Emollients can help the skin feel more comfortable if you have eczema, psoriasis, dry, or sensitive skin.

The term “humectant” as used herein includes a substance that draws water into the skin, hair, or nails. In the skin, this may come from the deeper layers, or from the air if it is humid enough. Humectants are useful for adding hydration without feeling heavy or oily. Humectants include ingredients such as glycerin, urea, AHAs, and hyaluronic acid.

### EXAMPLES

The following examples are included to demonstrate some aspects of the present inventive concept. However, those of skill in the art should, in light of the present inventive concept, appreciate that many changes can be made in the specific aspects which are disclosed and still obtain a like or similar result without departing from the spirit and scope of the present inventive concept.

Example of Topical Formulation I is provided in Table 2.

TABLE 2
Topical Formulation I

| Ingredients | Amount % (wt/wt) |
|---|---|
| Water | about 80% to about 90% |
| Bakuchiol | about 0.01% to about 1% |
| Coenochloris signiensis Extract | about 0.01% to about 1% |
| Excipient | about 0.1% to about 10% |

Table 3 provides an example of topical formulation II according to one aspect of the disclosure.

TABLE 3
Topical Formulation II

| Ingredients | Amount % (wt/wt) |
|---|---|
| Water | about 80% to about 90% |
| Cetearyl alcohol | about 1% to about 10% |
| Caprylic/capric triglyceride | about 1% to about 10% |
| Glyceryl stearate | about 1% to about 10% |
| Cocos nucifera oil | about 1% to about 10% |
| Glycerin | about 1% to about 10% |
| Aloe barbadensis leaf juice | about 1% to about 10% |
| Maltodextrin | about 1% to about 10% |
| Coconut alkanes | about 1% to about 10% |
| Butyrospermum parkii nut extract | about 1% to about 10% |
| Bakuchiol | about 0.01% to about 1% |
| Coco-caprylate/caprate | about 0.01% to about 1% |
| Coenochloris signiensis extract | about 0.01% to about 1% |
| Lecithin | about 0.01% to about 1% |
| Carbomer | about 0.01% to about 1% |
| Sodium phytate | about 0.01% to about 1% |
| Xanthan gum | about 0.01% to about 1% |
| Ethylhexyglycerin | about 0.01% to about 1% |
| Tocopheryl acetate | about 0.01% to about 1% |
| Phenoxyethanol | about 0.01% to about 1% |
| Sodium hydroxide | about 0.01% to about 1% |

Table 4 further provides an example of a topical formulation III according to another aspect of the disclosure.

TABLE 4
Topical Formulation III

| Ingredients | Amount % (wt/wt) |
|---|---|
| Water | about 80% to about 90% |
| Cetearyl alcohol | about 1% to about 10% |
| Caprylic/capric triglyceride | about 1% to about 10% |
| Glyceryl stearate | about 1% to about 10% |
| Cocos nucifera oil | about 1% to about 10% |
| Glycerin | about 1% to about 10% |
| Aloe barbadensis leaf juice | about 1% to about 10% |
| Maltodextrin | about 1% to about 10% |
| Coconut alkanes | about 1% to about 10% |
| Butyrospermum parkii nut extract | about 1% to about 10% |
| Bakuchiol | about 0.01% |
| Coco-caprylate/caprate | about 0.01% to about 1% |
| Coenochloris signiensis extract | about 0.01% |
| Lecithin | about 0.01% to about 1% |
| Carbomer | about 0.01% to about 1% |
| Sodium phytate | about 0.01% to about 1% |
| Xanthan gum | about 0.01% to about 1% |
| Ethylhexyglycerin | about 0.01% to about 1% |
| Tocopheryl acetate | about 0.01% to about 1% |
| Phenoxyethanol | about 0.01% to about 1% |
| Sodium hydroxide | about 0.01% to about 1% |

What is claimed is:

1. A topical composition comprising:
a. a natural retinol alternative;
b. Coenochloris signiensis extract;
c. at least one excipient; and
d. at least one solvent.

2. The topical composition of claim 1, wherein the natural retinol alternative is present in the composition at a range of about 0.01% (wt/wt) to about 1% (wt/wt).

3. The topical composition of claim 1, wherein the natural retinol alternative is bakuchiol.

4. The topical composition of claim 1, wherein the Coenochloris signiensis extract is present in the composition at a range of about 0.01% (wt/wt) to about 1% (wt/wt).

5. The topical composition of claim 1, wherein the at least one excipient or solvent is present in the composition at about 1% (wt/wt) to about 90% (wt/wt).

6. The topical composition of claim 5, wherein the at least one excipient is cetearyl alcohol, glycerin, carbomer, xanthan gum, or any combination thereof.

7. The topical composition of claim 1, wherein the at least one solvent is water.

8. The topical composition of claim 1, wherein the composition further comprises at least one skin conditioner and/or at least one emollient selected from caprylic/capric triglyceride, glyceryl stearates, Cocos nucifera oil, Aloe barbadensis leaf juice, maltodextrin, coconut alkanes, Butyrospermum parkii nut extract, coco-caprylate/caprate, lecithin, ethylhexylglycerin, tocopheryl acetate, and any combination thereof.

9. The topical composition of claim 8, wherein the at least one skin conditioner and/or at least one emollient is present in the composition at about 1% (wt/wt) to about 15% (wt/wt).

10. The topical composition of claim 1, wherein the composition further comprises phenoxyethanol preservative.

11. The topical composition of claim 10, wherein the at least one preservative is present in the composition at about 0.01% (wt/wt) to about 1% (wt/wt).

12. The topical composition of claim 1, wherein the composition further comprises at least sodium hydroxide buffer.

13. The topical composition of claim 12, wherein the buffer is present at in the composition at about 0.1% (wt/wt) to about 5% (wt/wt).

14. The topical composition of claim 1, wherein the composition further comprises sodium phytate chelator.

15. The topical composition of claim 14, wherein the chelator is present in the composition at about 0.01% (wt/wt) to about 1% (wt/wt).

16. A topical composition comprising water, cetearyl alcohol, caprylic/capric triglyceride, glyceryl stearate, Cocos nucifera oil, glycerin, Aloe barbadensis leaf juice, maltodextrin, coconut alkanes, Butyrospermum parkii nut extract, bakuchiol, coco-caprylate/caprate, Coenochloris signiensis extract, lecithin, carbomer, sodium phytate, xanthan gum, ethylhexylglycerin, tocopheryl acetate, phenoxyethanol, sodium hydroxide.

17. A topical composition comprising:
Water: about 80 to about 90% (wt/wt);
Cetearyl alcohol: about 1% (wt/wt) to about 10% (wt/wt);
Caprylic/Capric Triglyceride: about 1% (wt/wt) to about 10% (wt/wt);
Glyceryl Stearate: about 1% (wt/wt) to about 10% (wt/wt);
Cocos nucifera oil: about 1% (wt/wt) to about 10% (wt/wt);
Glycerin: about 1% (wt/wt) to about 10% (wt/wt);
Aloe barbadensis leaf juice: about 1% (wt/wt) to about 10% (wt/wt);
Maltodextrin: about 1% (wt/wt) to about 10% (wt/wt);
Coconut alkanes: about 1% (wt/wt) to about 10% (wt/wt);
Butyrospermum parkii nut extract: about 1% (wt/wt) to about 10% (wt/wt);
Bakuchiol: about 0.01% (wt/wt) to about 1% (wt/wt);
Coco-Caprylate/Caprate: about 0.01% (wt/wt) to about 1% (wt/wt);
Coenochloris signiensis extract: about 0.01% (wt/wt) to about 1% (wt/wt);
Lecithin: about 0.01% (wt/wt) to about 1% (wt/wt);
Carbomer: about 0.01% (wt/wt) to about 1% (wt/wt);
Sodium phytate: about 0.01% (wt/wt) to about 1% (wt/wt);
Xanthan gum: about 0.01% (wt/wt) to about 1% (wt/wt);
Ethylhexyglycerin: about 0.01% (wt/wt) to about 1% (wt/wt);
Tocopheryl acetate: about 0.01% (wt/wt) to about 1% (wt/wt) Phenoxyethanol: about 0.01% (wt/wt) to about 1% (wt/wt);
Sodium hydroxide: about 0.01% (wt/wt) to about 1% (wt/wt).

18. The topical composition of claim 17, wherein the composition is formulated as a moisturizing cream.

19. A method of improving a condition of the skin of a subject in need thereof comprising, applying to the skin of the subject the topical composition of claim 17; wherein applying the topical composition (a) prevent, reduce, or treat dryness, fine lines, aging of the skin cells, crow's feet, wrinkles, moisture loss, hyperpigmentation, uneven skin tone, crepe skin, collagen loss, oxidation, skin redness, roughness, free radical formation, UV damage, photo-damage, blue light damage, or any combination thereof; or (b) enhance hydration, clarity, radiance, passage for absorption, skin brightness, healthy glow, even tone, elasticity, firmness, smoothness or any combination thereof.
